(19) United States
(12) Patent Application Publication
(10) Pub. No.: US 2002/0147187 A1
(43) Pub. Date: Oct. 10, 2002

(54) 1,2-diphenyl-1-naphthyl ethene derivatives, analogs and use thereof

(76) Inventors: Jonathan Martin Schmidt, Elora (CA); Julie Mercure, Guelph (CA); Shuguang Zhu, Seattle, WA (US); John Whelan, Toronto (CA); Natalie Lazarowych, Richmond Hill (CA)

Correspondence Address: FULBRIGHT & JAWORSKI L.L.P., A REGISTERED LIMITED LIABILITY PARTNERSHIP, SUITE 2400, 600 CONGRESS AVENUE, AUSTIN, TX, 78701, US

(21) Appl. No.: 09/934,260

(22) Filed: Aug. 21, 2001

Related U.S. Application Data
(60) Provisional application No. 60/270,199, filed on Feb. 22, 2001.

(51) Int. Cl. C07D 265/30; A61K 31/535; A61K 31/55; A61K 31/4465; A61K 31/40; A61K 31/277; A61K 31/137

(52) U.S. Cl. 514/212.01; 514/238.8; 514/319; 514/428; 514/520; 514/651; 540/609; 544/170; 546/205; 548/570; 558/418; 564/347

(57) ABSTRACT

Molecules demonstrating anti-proliferative effects against epithelial cancer cell lines, human estrogen-dependent cancer cells and endothelial cells are disclosed. The molecules are intended for use in therapeutic preparations for the treatment of various cancers. The compounds specified are 1,2-diphenyl-1-naphthyl ethene derivatives.

### FIELD OF THE INVENTION

The present invention relates to a series of new chemical agents that demonstrate anti-proliferative effects against either all or a combination of, epithelial cancer cells, human estrogen-dependent cancer cells and endothelial cells, thus demonstrating the potential to treat a variety of disease states. More particularly, the present invention relates to molecules that demonstrate binding affinity for the estrogen receptor and anti-proliferative capabilities against epithelial cancer cell lines, human estrogen-dependent cancer cells and endothelial cells. The present invention also relates to their method of synthesis and their applications in treating a variety of disease states.

### BACKGROUND OF THE INVENTION

Cancer is a disease state characterized by the uncontrolled proliferation of genetically altered tissue cells. There have been several chemotherapeutic approaches developed to target cancer. These include alkylating and anti-mitotic agents, anti-metabolites and anti-tumor antibiotics. Such therapeutic agents act preferentially on rapidly proliferating cells such as cancer cells.

Hormonal therapy using anti-estrogens or anti-androgens constitutes another method of treating cancer. One approach has been to prevent estrogen biosynthesis using inhibitors of aromatase enzymes, which are responsible for the conversion of androgens to estrogens. Alternatively, estrogen activity may be interrupted at the receptor level using estrogen antagonists.

The involvement of estrogens in the development and progression of breast cancer has been known for over 100 years. In normal breast tissue, only 6% of the mammary epithelial cells express estrogen receptors (McDonnell et al. Ann. N. Y. Acad Sci 1996; 121-37), whereas over 60% of primary breast tumors are estrogen receptor positive and are dependent on estrogen for growth. However, it has been documented that other agents (e.g. growth factors) can activate estrogen receptors in the absence of estrogen (Pareczyk and Schneider, J. Cancer Res. Clin. Oncol. 1996; 122:383-96). As a result, blocking activity at the estrogen receptor is a potentially more effective therapeutic strategy than inhibiting estrogen biosynthesis.

Tamoxifen, a triphenylethene derivative, is the most widely used anti-estrogen for the treatment of breast cancer. It is predominantly used as first-line therapy in metastatic breast cancer to prolong survival. Unfortunately, resistance to tamoxifen usually develops within 15 months of therapy initiation (Howell et al., Lancet 1995; 345:29-30). Nevertheless, the clinical efficacy of tamoxifen as a hormonal therapy for many types of breast cancer has led to the search for more potent estrogen receptor antagonists.

In addition to treating breast cancer, the activity of endogenously produced estrogens can modulate the course of many estrogen-dependent diseases. Several new anti-estrogens including toremifene, droloxifene, idoxifene, TAT-59 and raloxifene are currently being evaluated in the laboratory and in the clinic for the treatment of estrogen-related disease states (Gradishar and Jordan, J. Clin. Oncol. 1997; 15(2):840-52). There has been considerable concern regarding the long-term use of tamoxifen due to an increase in incidences of endometrial cancer, deep venous thrombosis and pulmonary embolism in patients receiving the therapy (Rauschning and Pritchard, Breast Cancer Res. Treat 1994; 31:83-94). Other, more common side effects include, hot flushes, vaginal bleeding and blurred vision (Nicholson, Bailliere, Tindall, 1987:60-87). Despite these side effects, results from one clinical study have demonstrated the utility of tamoxifen in the prevention of breast cancer in women at high risk of developing the disease (Fisher et al., J. of the Nat'l Cancer Inst. 1998; Vol. 90; No.18; 1371-1388) and the FDA has approved it for prophylactic use.

It has been suggested that the partially agonistic properties of some anti-estrogens are responsible for both their side-effect profile and the development of resistance to therapy (Nicholson et al., Ann. N. Y Acad. Sci. 1996; 784:325-35). Partial agonists are compounds for which the balance in the expression of antagonistic and agonistic activity depends on the dose administered, as well as on the species and target organ studied. More specifically, differences in agonistic/antagonistic responses depend on the presence of cell-specific proteins that can act as co-activators or transcription factors (Mitlak and Cohen, Horm. Res. 1997; 48:155-63). In vitro and in vivo experiments have suggested that the agonistic properties of some anti-estrogens may become dominant through the course of therapy. This has been demonstrated in clinical settings where 10-30% of tamoxifen-resistant patients showed improvement of their condition after withdrawal from tamoxifen therapy (Parczyk and Schneider, J. Cancer Res. Clin. Oncol. 1996; 122:383-96).

“Pure” anti-estrogens are compounds that have exclusively antagonistic properties and lead to the formation of inactive ligand-receptor complexes. In contrast to partial agonists, that stimulate the expression of estrogen receptors, pure anti-estrogens cause a down-regulation of cellular receptor protein levels (Parczyk and Schneider, J. Cancer Res. Clin. Oncol. 1996; 122:383-96). Since the estrogen receptor is activated through estrogen-independent factors, the reduction in estrogen receptor levels obtained with pure anti-estrogens may offer clinical advantages over partial agonists and aromatase inhibitors. Clinical trials with pure anti-estrogens have shown efficacy against tamoxifen-resistant breast cancers, where approximately two-thirds of tamoxifen-resistant patients responded to ICI 182780 (faslodex) and no significant adverse effects were observed (England and Jordan, Oncol. Res. 1997; 9:397-402).

Many studies performed to date have suggested that anti-estrogens with partial agonistic activity, have positive effects on cardiovascular and skeletal systems. For example, tamoxifen lowers total and LDL cholesterol, lowers lipoprotein (A) and preserves bone mass in post-menopausal women undergoing breast cancer treatment (Mitlak and Cohen, Horm. Res. 1997; 48:155-63). Estrogens play an important role in the regulation and synthesis of lipids and therefore have a protective effect on the cardiovascular system. Following menopause, the risk of developing atheriosclerosis and coronary disorders, dramatically increases in women not undergoing hormone replacement therapy. In addition, estrogens are critically important in the maintenance of proper bone mass. As the circulating level of estrogen decreases, post-menopausal women experience an increase in the rate of bone turnover, resulting in net bone loss. Therefore, the observed positive effects of tamoxifen on skeletal and cardiovascular systems, may be related to agonistic activity through the estrogen receptor present in those tissues (Mitlak and Cohen, Horm. Res. 1997; 48:155-63).

Other partial agonists currently in development have demonstrated anti-estrogenic effects on reproductive tissues with increased protective effects or estrogenic activity on the skeletal and cardiovascular systems. These compounds are known as Selective Estrogen Receptor Modulators (SERMs). Examples of these include droloxifene, which is being developed as an anti-osteoporotic agent and raloxifene, which has been approved by the FDA for the prevention of osteoporosis in post-menopausal women.

Although anti-cancer agents fall into specific classifications, it is not uncommon for agents to act by multiple modes of action. For example, tamoxifen has been shown to have anti-proliferative activity on cancer cells and endothelial cells by an estrogen-independent mechanism (Coradini D. et al. Anticancer Research 1994; 14:1059-1064). Taxol, an anti-mitotic agent acting on microtubules, has also demonstrated anti-angiogenic properties, possibly by inducing apoptosis through Bcl-2 phosphorylation. The fact that some anti-estrogens have demonstrated anti-angiogenic properties is of particular interest to many in this field of research.

Angiogenesis, the formation of new blood vessels, is a fundamental biological process involved in wound healing, tissue regeneration, embryogenesis and the female reproductive cycle (Colville-Nash P. R. and Willoughby D. A., Molecular Medicine Today, Jan. 14-23, 1997). Blood vessel walls are formed by endothelial cells that have the ability to divide and migrate under specific stimuli, such as growth factors. The creation of new blood vessels follows a specific set of tightly regulated steps (Risau W., Nature, 1997; 386:671-674). Briefly, endothelial cells are stimulated by factors secreted by surrounding cells and secrete enzymes such as matrix metalloproteinases that break down the extra-cellular matrix and basement membrane, thus creating a space for the endothelial cells to migrate into and establish themselves. The endothelial cells then organize into hollow tubes that eventually form a new vascular network of blood vessels, providing surrounding cells with nutrients and oxygen and the ability to eliminate toxic metabolic waste products. Under normal physiological conditions, endothelial cells are dormant unless triggered to proliferate in localized parts of tissues. Many diseases are associated with the inappropriate proliferation of endothelial cells. Some examples include arthritis, psoriasis, atheriosclerosis, diabetic retinopathy and cancer (Jain R. K. et al. Nature Medicine 1997; 3:1203-1207).

In order for a tumor to grow beyond a few million cells, typically more than 1 or 2 mm³ in volume, an increase in vascularization is required (Twardowski P. and Gradishar W. J., Current Opinion in Oncology, 1997; 9:584-589). Clinically, tumors that are highly vascularized are the most aggressive (metastatic) and difficult to treat. It is also known that tumor cells produce and secrete the factors necessary for the angiogenesis process (Colville-Nash P. R. and Willoughby D. A., Molecular Medicine Today, Jan. 14-23, 1997). It is widely held that agents inhibiting angiogenesis through direct competition with angiogenic factors or by some other mechanism, would have a major clinical benefit in the treatment of many types of cancer and other diseases associated with inappropriate angiogenesis.

Many therapeutic agents are being developed based on a variety of targeting strategies. One strategy is the use of natural inhibitors of angiogenesis, such as angiostatin and endostatin (O'Reilly M. S. et al. Cell, 1994; 79:315-328). Another strategy is the use of agents that block the receptors required for stimulating angiogenesis, such as antagonists to the vitronectin receptor (Keenan R. M. et al. Journal of Medicinal Chemistry, 1997; 40:2289-2292). Yet a third strategy is the inhibition of specific enzymes which allow new blood vessels to invade surrounding tissues, for example, inhibitors of matrix metalloproteinases.

Angiogenesis is an attractive therapeutic target for cancer treatment due to its selective mode of action. Blood vessels in growing tumors are rapidly proliferating and being replaced, whereas blood vessels in most normal tissues are static. This rapid turnover is believed to be the physiological difference that will allow the selective targeting of blood vessels in tumors by anti-angiogenic agents. Anti-angiogenesis is also less likely to pose a drug resistance problem as compared to conventional chemotherapy. Tumor cells are prone to mutations that render them resistant to standard chemotherapy. Since anti-angiogenic agents target normal but rapidly proliferating endothelial cells that are not genetically unstable, resistance to anti-angiogenic agents is not a major concern.

Anti-angiogenic therapy will likely be very effective at suppressing tumor growth by denying tumors a blood supply. However, anti-angiogenic therapy may prove more effective in combination with other therapies aimed directly at tumor cells. Chemical agents that demonstrate both anti-angiogenic and tumor directed properties would obviously be greatly desired.

There thus remains a need to develop a series of new chemical agents that demonstrate anti-proliferative effects against either all or a combination of, epithelial cancer cells, human estrogen-dependent cancer cells and endothelial cells, thus demonstrating the potential to treat a variety of disease states.

The present invention seeks to meet these and other needs.

The present description refers to a number of documents, the content of which is herein incorporated by reference in their entirety.

### SUMMARY OF THE INVENTION

The present invention relates to a series of new chemical agents that demonstrate anti-proliferative effects against either all or a combination of, epithelial cancer cells, human estrogen-dependent cancer cells and endothelial cells, thus demonstrating the potential to treat a variety of disease states.

The present invention more particularly relates to molecules that are non-steroidal derivatives of 1,2-diphenyl-1-naphthyl ethene and analogs thereof and to their synthesis. Furthermore, the invention relates to the demonstration that such molecules act as anti-cancer agents.

The present invention relates to a therapeutic composition of molecules useful in the treatment of cancer and other diseases, characterized by the undesired proliferation of endothelial or epithelial cells such as, but not limited to, pathological tissue growth, psoriasis, diabetic retinopathy, rheumatoid arthritis, hemangiomas, solid tumor formation and other malignancies.

The present invention relates to a therapeutic anti-estrogen composition useful in the treatment of estrogen-related diseases. These diseases include, but are not limited to, breast cancer, uterine cancer, ovarian cancer, osteoporosis, cardiovascular diseases, premenstrual syndrome, uterine fibroma, endometriosis, precocious puberty, vasomotor symptoms associated with menopause, atrophic vaginitis, CNS disorders (including Alzheimer's), infertility, glaucoma and elevated blood serum cholesterol.

As well, the present invention relates to non-steroidal anti-estrogens having good affinity for estrogen receptors, but substantially lacking undesirable agonistic activity with respect to the estrogen receptors in reproductive tissues.

In accordance with one embodiment of the present invention, there is provided a pharmaceutical composition comprising a therapeutically effective amount of an anti-cancer molecule specified herein. As used herein, the terms R¹, R², R³ and R⁴ refer to effective functional groups whose location on the 1,2-diphenyl-1-naphthyl ethene backbone is illustrated by Formula I below:

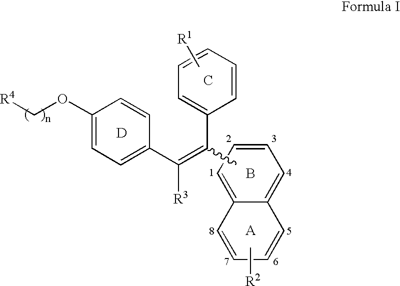

The B-ring of the naphthyl group may be linked to ethene at either the 1-position or the 2-position, as depicted by Formula I, without materially affecting the scope of the present invention.

Certain preferred substituents for R¹ include, but are not limited to hydroxyl, methoxy, ethoxy and esters. The R¹ substituent is preferable in the para position, however in other embodiments of the invention it is either in the ortho or meta position on the C-ring.

Certain preferred substituents for R² include, but are not limited to hydroxyl, methoxy, ethoxy and esters. The R² substituent is preferable in the 6-position on the A-ring, however in other embodiments of the invention it is in the 5, 7 or 8-position on the A-ring.

Certain preferred substituents for R³ include, but are not limited to hydrogen, methyl, ethyl and cyano.

Certain preferred substituents for R⁴ include, but are not limited to 1-pyrrolidinyl or 1-piperidinyl, methyl-1-pyrrolidinyl, dimethyl-1-pyrrolidinyl, 4-morpholino, dimethylamino, diethylamino, diisopropylamino, or 1-hexamethyleneimino and in each case n is an integer from 1 to 4. The nitrogen atom contained in the pyrrolidine and piperidine ring systems is predominantly protonated at physiological pH. Furthermore, the incorporation of the nitrogen atom into a ring system such as in pyrrolidine or piperidine, acts to prevent potential toxicity associated with N-dealkylation, which has been shown to readily occur with the dimethylaminoethoxy side chain of tamoxifen (Grandishar and Jordan, J. Clin. Oncol. 1997; 15(2): 840-52).

Certain preferred embodiments, in which a 6-hydroxynaphthyl group is positioned at the C-1 carbon of the ethene backbone and in which a 4-[2-(1-pyrrolidinyl)ethoxy]phenyl group is positioned at the C-2 carbon of the ethene backbone, may be either in the Z or E-configuration.

In accordance with the present invention, there is therefore provided a compound of Formula I, comprising an ethene backbone, and A, B, C and D rings, or a pharmaceutically acceptable salt or ester thereof,

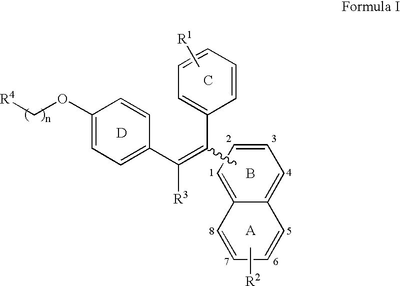

wherein R¹ represents a substituent selected from the group consisting of hydroxyl, methoxy, ethoxy and esters; wherein R² represents a substituent selected from the group consisting of hydroxyl, methoxy, ethoxy and esters; wherein R³ represents a substituent selected from the group consisting of hydrogen, methyl, ethyl and cyano; wherein R⁴ represents a substituent selected from the group consisting of 1-pyrrolidinyl, 1-piperidinyl, methyl-1-pyrrolidinyl, dimethyl-1-pyrrolidinyl, 4-morpholino, dimethylamino, diethylamino, diisopropylamino and 1-hexamethyleneimino; wherein “n” is an integer from 1 to 4; wherein the B-ring is connected at the 1-position or the 2-position to the ethene backbone; wherein the R¹ substituent is located at the ortho, meta or para-position on the C-ring; wherein the R² substituent is located at either the 5, 6, 7 or 8-position on the A-ring, and wherein the ethene backbone has the E-configuration or the Z-configuration with respect to the B-ring and the D-ring.

In accordance with the present invention, there is also provided a process for the preparation of a compound of Formula I, involving the reaction of a molecule of Formula 1.12;

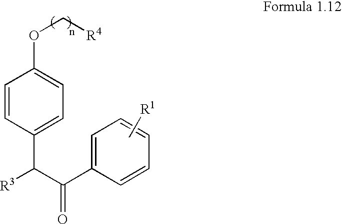

wherein R¹ represents a substituent selected from the group consisting of hydroxyl, methoxy, ethoxy and esters; wherein R³ represents a substituent selected from the group consisting of hydrogen, methyl, ethyl and cyano; wherein R⁴ represents a substituent selected from the group consisting of 1-pyrrolidinyl, 1-piperidinyl, methyl-1-pyrrolidinyl, dimethyl-1-pyrrolidinyl, 4-morpholino, dimethylamino, diethylamino, disopropylamino and 1-hexamethyleneimino; wherein “n” is an integer from 1 to 4; and wherein the R¹ substituent is located at the ortho, meta or para-position; with a reagent mixture composed of

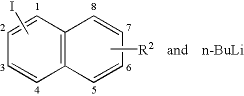

wherein R² represents a substituent selected from the group consisting of hydroxyl, methoxy, ethoxy and esters; wherein I is located at the 1- or 2-position, and wherein R² is located at the 5, 6, 7 or 8-position; followed by the treatment of the reaction mixture with hydrochloric acid and recovering the compound of Formula I from the reaction mixture.

In accordance with the present invention, there is also provided a process for the preparation of a compound of Formula I (as shown above), wherein R¹ and R² are hydroxyl groups; wherein R³ is a methyl group; wherein R⁴is a pyrrolidine group; wherein “n” is 2; wherein R¹ is located at the para-position on the C-ring; wherein R² is located at the 6-position on the A-ring, and wherein the B-ring is linked to the ethene backbone at the 1-position, as depicted by the compound of Formula II;

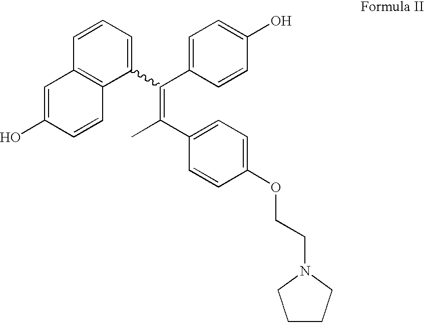

involving the reaction of a compound of Formula 1.5;

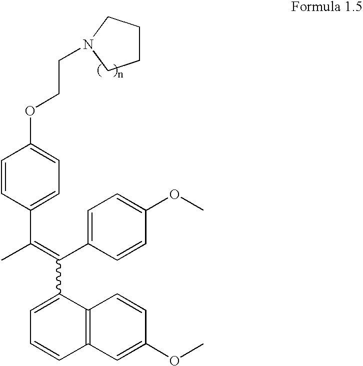

wherein n=1; with a reagent mixture composed of HBr or BBr₃ followed by the recovery of the compound of Formula II from the reaction mixture.

In accordance with the present invention, there is provided an additional process for the preparation of a compound of Formula II (as shown and described above), involving the reaction of a molecule of Formula 1.8;

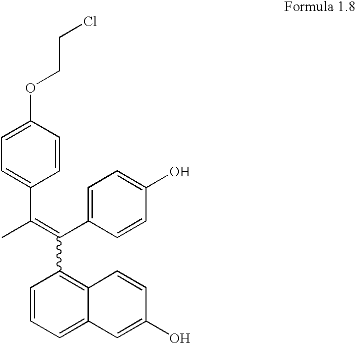

with

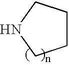

wherein n=1 and recovering the compound of Formula II from the reaction mixture.

In accordance with the present invention, there is also provided a process for the preparation of a compound of Formula I (as shown above), wherein R¹ and R² are methoxy groups; wherein R³ is a methyl group; wherein R⁴ is a pyrrolidine group; wherein “n” is 2; wherein R¹ is located at the para-position on the C-ring; wherein R² is located at the 6-position on the A-ring, and wherein the B-ring is linked to the ethene backbone at the 1-position, as depicted by the compound of Formula III;

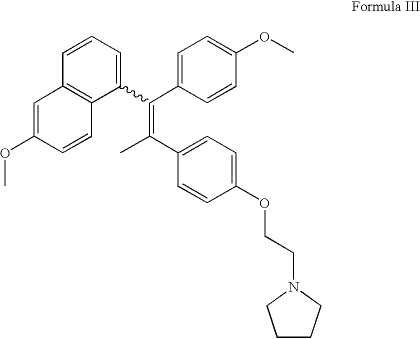

involving the reaction of a compound of Formula 1.4;

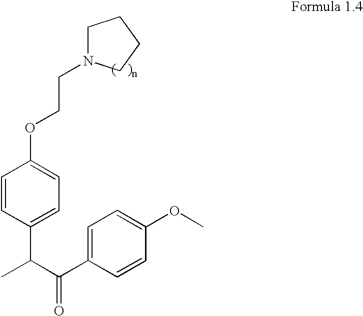

wherein n=1; with a reagent mixture composed of

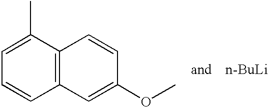

followed by treatment of the reaction mixture with hydrochloric acid, and recovering the compound of Formula III from the reaction mixture.

In accordance with the present invention there is provided a pharmaceutical composition comprising the compound represented by Formula I and at least one pharmaceutically acceptable carrier.

In accordance with the present invention there is provided a process for the preparation of anti-cancer agents, represented by Formula I, involving the reaction of a molecule of Formula 1.12, as previously defined, with a reagent mixture composed of

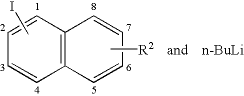

wherein R² represents a substituent selected from the group consisting of hydroxyl, methoxy, ethoxy and esters; wherein I is located at the 1- or 2-position, and wherein R² is located at the 5, 6, 7 or 8-position; followed by the treatment of the reaction mixture with hydrochloric acid and recovering the anti-cancer agents of Formula I from the reaction mixture.

In accordance with the present invention there is provided a method of treating cancer involving the administration of a therapeutically effective amount of a compound of Formula I to a patient in need thereof.

Unless defined otherwise, the scientific and technological terms and nomenclature used herein have the same meaning as commonly understood by a person of ordinary skill. Generally, procedures such as recovering a-or more compounds from a reaction mixture are common methods used in the art. Such standard techniques can be found in reference manuals such as for example Gordon and Ford (The Chemist's Companion: A Handbook of Practical Data, Techniques and References, John Wiley & Sons, New York, N.Y., 1972).

The present description refers to a number of routinely used chemical terms. Nevertheless, definitions of selected examples of such terms are provided for clarity and consistency.

As used herein, the terminology “pharmaceutical composition” or “pharmaceutical formulation”, well known in the art, are used interchangeably.

As used herein, the terminology “recovering”, a desired compound or the like, well known in the art, refers to such a desired compound having been isolated from other components of a reaction mixture.

The present invention comprises the genus of compounds represented by Formula I, useful in the treatment of cancer and other diseases characterized by the undesired proliferation of endothelial or epithelial cells such as, but not limited to, pathological tissue growth, psoriasis, diabetic retinopathy, rheumatoid arthritis, hemangiomas, solid tumor formation and other malignancies as well as in the treatment and or prevention of a variety of disorders or conditions such as breast cancer, uterine cancer, ovarian cancer, bone tissue loss (osteoporosis), cardiovascular diseases, premenstrual syndrome, uterine fibroma, endometriosis, precocious puberty, vasomotor symptoms associated with menopause, atrophic vaginitis, CNS disorders (including Alzheimer's), infertility, glaucoma and elevated blood serum cholesterol.

It will be appreciated by those skilled in the art that reference herein to treatment extends to prophylactic treatment as well as to the treatment of established diseases or symptoms. It will be further appreciated that the amount of a compound of the invention required for use in treatment will vary with the nature of the condition being treated, the age and condition of the patient and will ultimately be at the discretion of the attending physician or medical practitioner. In general however, doses employed for adult human treatment will typically be in the range of 0.001 mg/kg to about 100 mg/kg per day. The desired dose may conveniently be presented in a single dose or as divided doses administered at appropriate intervals such as for example two, three, four or more sub-doses per day. It will be further appreciated by those skilled in the art that compounds of Formula I may be administered alone or in conjunction with standard tumor therapy, such as chemotherapy or radiation therapy.

The present invention also provides for novel pharmaceutical compositions of the compounds of Formula I. While it is possible that compounds of the present invention may be therapeutically administered as the raw chemical, it is preferable to present the active ingredient as a pharmaceutical formulation. Accordingly, the present invention further provides for pharmaceutical formulations comprising a compound of Formula I or a pharmaceutically acceptable salt or ester thereof together with one or more pharmaceutically acceptable carriers and, optionally, other therapeutic and/or prophylactic ingredients. The carrier(s) must be “acceptable” in the sense of being compatible with the other ingredients of the formulation and not be deleterious to the recipient thereof.

Formulations of the present invention, for the treatment of the indicated diseases, may be administered in standard manner, such as orally, parenterally, sublingually, transdermally, rectally or via inhalation. For oral administration the composition may take the form of tablets or lozenges formulated in a conventional manner. For example, tablets and capsules for oral administration may contain conventional excipients such as binding agents, (for example, syrup, accacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycollate) or wetting agents, such as sodium lauryl sulphate. The tablets may be coated according to methods well-known in the art.

Alternatively, the compounds of the present invention may be incorporated into oral liquid preparations such as aqueous or oily suspensions, solutions, emulsions, syrups or elixirs. Moreover, formulations containing these compounds may be presented as a dry product for constitution with water or other suitable vehicle before use. Such liquid preparations may contain conventional additives such as suspending agents such as sorbitol syrup, methyl cellulose, glucose/sugar syrup, gelatin, hydroxyethylcellulose, carboxymethyl cellulose, aluminum stearate gel or hydrogenated edible fats; emulsifying agents such as lecithin, sorbitan mono-oleate or acacia; non-aqueous vehicles (which may include edible oils) such as almond oil, fractionated coconut oil, oily esters, propylene glycol or ethyl alcohol; and preservatives such as methyl or propyl p-hydroxybenzoates or sorbic acid.

Such preparations may also be formulated as suppositories, e.g., containing conventional suppository bases such as cocoa butter or other glycerides. Compositions for inhalation can be typically provided in the form of a solution, suspension or emulsion that may be administered as a dry powder or in the form of an aerosol using a conventional propellant such as dichlorodifluoromethane or trichlorofluoromethane. Typical transdermal formulations comprise a conventional aqueous or non-aqueous vehicle, such as creams, ointments, lotions or pastes or are in the form of a medicated plaster, patch or membrane.

Additionally, compositions of the present invention may be formulated for parenteral administration by injection or continuous infusion. Formulations for injection may take such forms as suspensions, solutions, or emulsions in oily or aqueous vehicles, and may contain formulatory agents such as suspending, stabilizing and/or dispersing agents. Alternatively, the active ingredient may be in powder form for constitution with a suitable vehicle (e.g., sterile, pyrogen-free water) before use.

Compositions of the present invention may be formulated for nasal administration. Such formulations may comprise the selected compound of the present invention and a non-toxic pharmaceutically acceptable nasal carrier. Suitable non-toxic pharmaceutically acceptable nasal carriers for use in the compositions of the present invention will be apparent to those skilled in the art of nasal pharmaceutical formulations. Obviously the choice of suitable carriers will depend on the exact nature of the particular nasal dosage form desired, as well as on the identity of the active ingredient(s). For example, whether the active ingredient(s) are to be formulated into a nasal solution (for use as drops or spray), a nasal suspension, a nasal ointment or a nasal gel. Preferred nasal dosage forms are solutions, suspensions and gels, which contain a major amount of water (preferably purified water) in addition to the active ingredient(s). Minor amounts of other ingredients such as pH adjusters (e.g., a base such as NaOH), emulsifiers or dispersing agents (e.g. polyoxyethylene 20 sorbitan mono-oleate), buffering agents, preservatives, wetting agents and gelling agents (e.g. methylcellulose) may also be present. Also, a sustained release composition (e.g. a sustained release gel) can be readily prepared.

The composition according to the present invention may also be formulated as a depot preparation. Such long acting formulations may be administered by implantation (for example, subcutaneously or intramuscularly) or by intramuscular injection. Accordingly, the compounds of the present invention may be formulated with suitable polymeric or hydrophobic materials (such as an emulsion in an acceptable oil), ion exchange resins or as sparingly soluble derivatives or sparingly soluble salts.

The terms and descriptions used herein are preferred embodiments set forth by way of illustration only, and are not intended as limitations on the many variations which those of skill in the art will recognize to be possible in practicing the present invention. It is the intention that all possible variants whether presently known or unknown, that do not have a direct and material effect upon the way the invention works, are to be covered by the following claims.

### BRIEF DESCRIPTION OF THE DRAWINGS

Having thus generally described the invention, reference will now be made to the accompanying drawings, showing by way of illustration a preferred embodiment thereof and in which:

FIG. 1 shows the effects of the compound depicted by formula II on the proliferation of MCF-7 estrogen-dependent cancer cells;

FIG. 2 shows the effects of the compound depicted by formula II on the proliferation of four cancer cell lines;

FIG. 3 shows the effects of the compound depicted by formula III on the proliferation of four epithelial cancer cell lines;

FIG. 4 shows the effects of the compounds depicted by formula II and formula III on the proliferation of HUVEC cells; and

FIG. 5 shows the effects of the compounds depicted by formula II and formula III on the proliferation of BBCE cells.

Other objects, advantages and features of the present invention will become more apparent upon reading the following non-restrictive description of preferred embodiments with reference to the accompanying drawings, which are exemplary and should not be interpreted as limiting the scope of the present invention.

### DESCRIPTION OF THE PREFERRED EMBODIMENT

The present invention is further illustrated by a series of new chemical agents that demonstrate anti-proliferative effects against either all or a combination of, epithelial cancer cells, human estrogen-dependent cancer cells and endothelial cells, thus demonstrating the potential to treat a variety of disease states.

In one preferred embodiment of the chemical agents as described by Formula I, displaying anti-proliferative effects in cells, the A, B, C and D rings are aromatic, R¹ is hydroxy, R² is hydroxy, R³ is methyl, R⁴ is pyrrolidine and n=2. Preferably at least one embodiment is represented by the following Formula II, or a pharmaceutically acceptable salt or ester thereof:

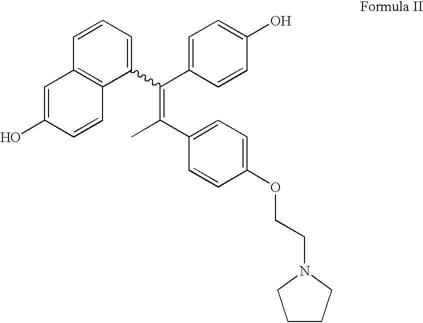

In another preferred embodiment of the invention, the A, B, C and D rings are aromatic, R¹ is methoxy, R² is methoxy, R³ is methyl, R⁴ is pyrrolidine and n=2. Preferably at least one embodiment is represented by the following Formula III, or a pharmaceutically acceptable salt or ester thereof:

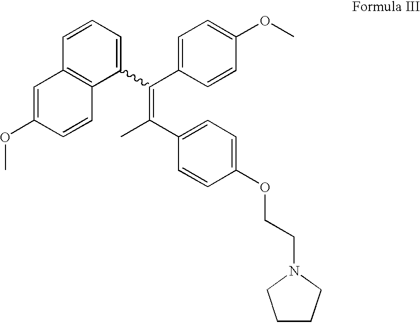

Set forth below is a preferred synthesis scheme for the preparation of certain preferred embodiments of the anti-cancer molecules in accordance with the invention. The synthetic steps set forth below are set forth merely by way of examples. Those skilled in the art will readily recognize alternative synthetic pathways and variations capable of producing a variety of the 1,2-diphenyl-1-naphthyl ethene derivatives, as represented by Formula I, in accordance with the present invention.

In the specification, the designations Z and E refer to the orientation of the 6-hydroxynaphthyl group on the C-1 carbon of the ethene backbone in respect to the 4-[2-(1-pyrrolidinyl)ethoxy]phenyl group located on the C-2 carbon of the same ethene backbone. The E and Z forms are distinguished from each other by proton nuclear magnetic resonance NOE (Nuclear Overhauser Effect) spectroscopy.

The present invention, which is defined by the claims, is illustrated in further detail by the following non-limiting examples.

### EXAMPLE 1

### A Process for the Preparation of Certain Preferred Embodiments for Compounds of Formula I is Depicted in Scheme 1

Compound 1.1 is made by alkylating anisole with 2-(4-nitrophenyl)propanoic acid at elevated temperatures, in the presence of a catalytic amount of polyphosphoric acid. A preferred temperature is 80° C. Catalytic hydrogenation of 1.1 over Pd/C (10%) for example, affords the amino compound 1.2. Diazotization of amine compound 1.2 followed by acid treatment affords the corresponding phenol 1.3, via a tetrafluoroborate salt intermediate. Alkylation of compound 1.3 with either 1-(2-chloroethyl)piperidine or 1-(2-chloroethyl)pyrrolidine in the presence of a base such as for example potassium carbonate, in a polar aprotic solvent such as dimethylformamide yields compound 1.4.

Following a halogen metal exchange reaction using n-butyllithium or magnesium metal, 1-iodo-6-methoxynaphthalene affords the corresponding lithium or magnesium reagent which is then reacted with amino ketone 1.4 to form an alcohol, which upon treatment with hydrochloric acid, yields a mixture of E/Z-alkene isomers 1.5. The preparation of 1-iodo-6-methoxynaphthalene is carried out according to the method disclosed by A. Butenandt and G. Schramm (Ber. (1935), 68, 2083).

In the final step, the cleavage of the two methoxy groups of 1.5 is carried out by either using a Lewis acid catalyst such as boron tribromide at −78° C., or with hydrogen bromide in acetic acid at elevated temperatures (80-100° C.), to provide the target compounds of Formula I.

Alternatively, compounds of Formula I can also be prepared by the alkylation of substrate 1.3 with 1,2-dicloroethane in the presence of a base such as for example sodium hydroxide and tetrabutylammonium hydrogen sulfate, to provide 1.6. The corresponding lithium or magnesium reagent derived from 1-iodo-6-methoxynaphthalene is reacted with 1.6 to form an alcohol, which upon treatment with hydrochloric acid affords a mixture of E/Z-alkene isomers 1.7. After cleavage of the two methoxy groups of 1.7, as previously described, the cis and trans isomers of 1-8 can be separated and isolated. The final step involves the nucleophilic substitution of the chlorine atom of the E-isomer of 1.8 by either pyrrolidine or piperidine in a protic solvent such as ethanol with heat to afford the target compounds of Formula I.

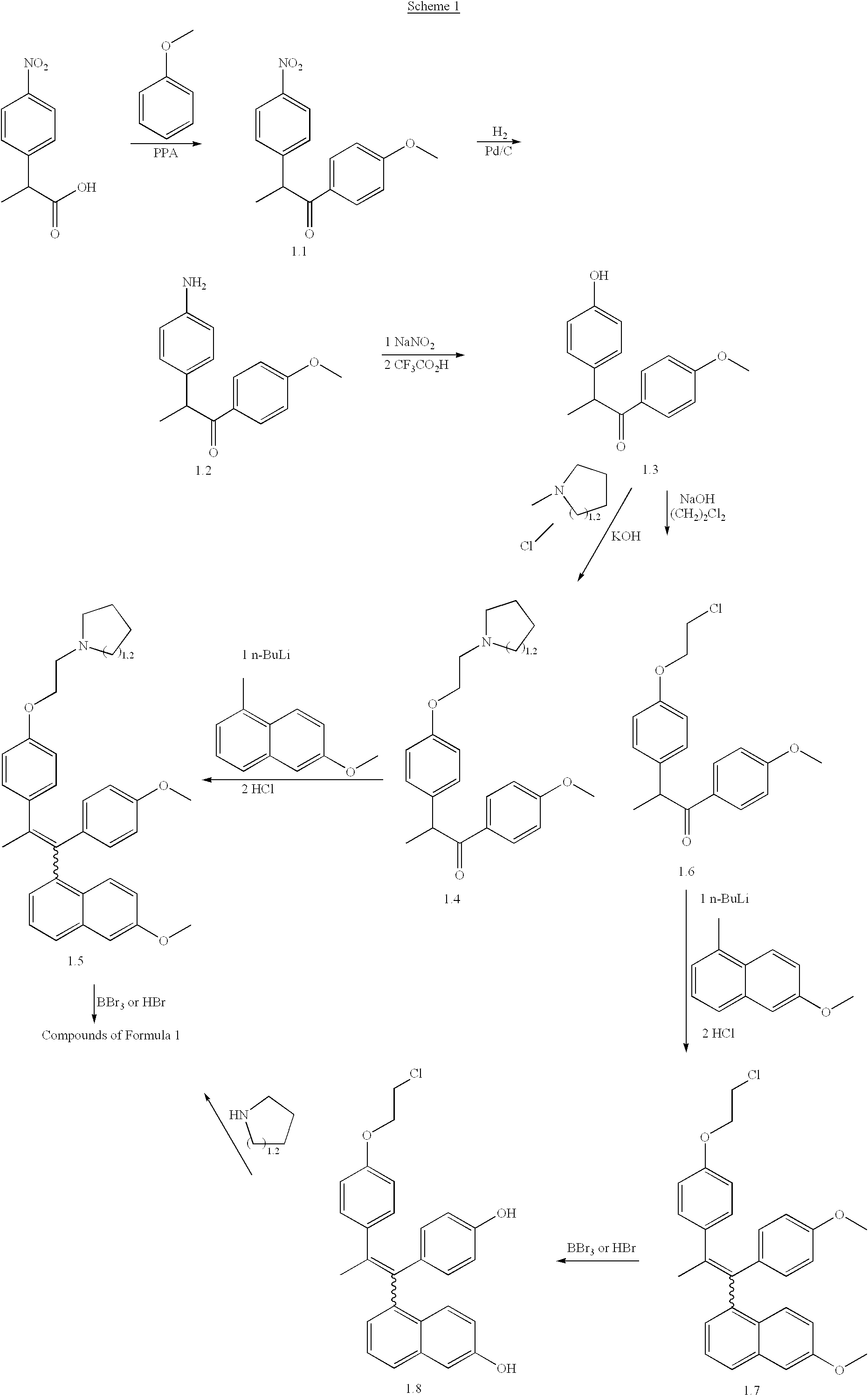

### EXAMPLE 2

### A General Process for the Preparation of Compounds of Formula I is Depicted in Scheme 2

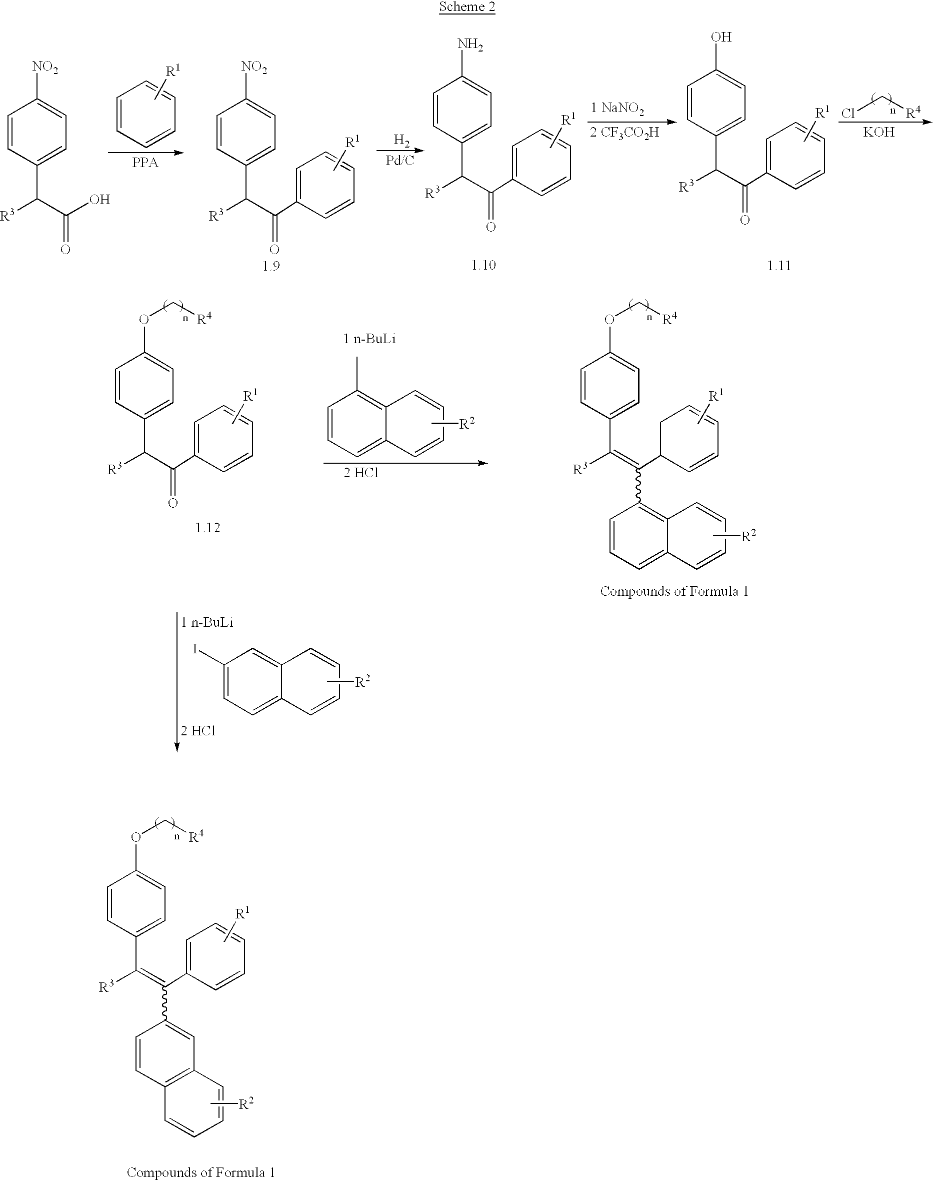

### EXAMPLE 3

### Synthesis of (E)-5-[1-(4-hydroxyphenyl)-2-[4-(2-pyrrolidin-1-yl-ethoxy)-phenyl]-propenyl]-naphthalen-2-ol

Step A 1-(4-methoxyphenyl)-2-(4-nitrophenyl)-propan-1-one

2-(4-nitrophenyl)propanoic acid (11.1 g, 57 mmol) and anisole (5.8 g, 62 mmol) were mixed with polyphosphoric acid (8.0 g) and heated to 80° C. for 3 h under Ar. The reaction mixture was cooled to room temperature, water (600 ml) was added and the mixture extracted three times with ethyl acetate (100 ml) to yield 13.5 g (83%) of the title compound. ¹H-NMR (400 MHz, CDCl₃) δ_{H} 1.58 (3H, d, CH₃), 3.85 (3H, s, OCH₃), 4.80 (1H, q, CH), 6.90 (2H, d, ArH), 7.49 (2H, d, ArH), 7.94 (2H, d, ArH), 8.17 (2H, d, ArH).

Step B: 2-(4-aminophenyl)-1-(4-methoxyphenyl)-propan-1-one

The compound from Step A (10.1 g, 35.4 mmol) was dissolved in a solvent system composed of dioxane (200 ml) and acetic acid (5.0 ml), to which was then added 10% Pd/C (0.45 g). The reaction mixture was flushed with Ar and subsequently shaken under H₂ (30 psi) at room temperature. The reaction was extracted three times with ethyl acetate (350 ml) and the crude product chromatographed on silica gel with 7% CH₃OH/CH₂Cl₂ to yield 7.8 g (86%) of the title compound. ¹H-NMR (400 MHz, CDCl₃) δ_{H} 1.48 (3H, d, CH₃), 3.79 (3H, s, OCH₃), 4.53 (1H, q, CH), 6.61-7.93 (8H, ArH).

Step C: 2-(4-hydroxyphenyl)-1-(4-methoxyphenyl)-propan-1-one

The compound from Step B (45 g, 0.176 mol) was added to concentrated HCl (200 ml) and water (150 ml) and cooled to 0° C. in an ice/salt bath. Sodium nitrite (13.4 g, 0.195 mol) was added and the reaction stirred for 30 min. Cold sodium tetrafluoroborate (29.0 g, 0.265 mol) in water (80 ml) was then added and the mixture stirred for an additional 2 h in the ice/salt bath. The solid was collected, washed with Et₂O and dried under vacuum overnight. The dried solid was then added under Ar to cooled trifluoroacetic acid (0-5° C.) (300 ml), containing K₂CO₃ (12.5 g, 0.090 mol) and the reaction mixture refluxed for 24 h. The reaction mixture was treated with water (1 l) and stirred for 2 h. The crude product was separated from the aqueous layer and precipitate, and chromatographed on silica gel using 7% CH₃OH/CH₂Cl₂ to yield 33.7 g (74.8%) of the title compound. ¹H-NMR (400 MHz, DMSO-d6) δ_{H} 1.34 (3H, d, CH₃), 3.83 (3H, s, OCH₃), 4.76 (1H, q, CH), 6.67-7.96 (8H, ArH), 9.31 (1H, s, OH).

Step D: 2-[4-(2-chloroethoxy)phenyl]-1-(4-methoxyphenyl)-propan-1-one

The compound from Step C (4.0 g, 15.6 mmol) was dissolved in 1,2-dichloroethane (25 ml). Tetrabutylammonium hydrogen sulfate (0.24 g, 0.7 mmol) and 3M NaOH (20 ml) were added and the reaction mixture refluxed for 21 h. The crude mixture was then extracted with Et₂O (2×30 ml) and washed with 1 M HCl (30 ml) and H₂O (2×30 ml). The crude product was chromatographed on silica gel with hexane/ethyl acetate (5:1) to give 3.8 g (77%) of the title compound. ¹H-NMR (400 MHz, CDCl₃) δ_{H} 1.52 (3H, d, CH₃), 3.80 (2H, m, ClCH₂), 3.85 (3H, s, OCH₃), 4.20 (2H, t, CH₂O ), 4.63 (1H, q, CH), 6.88-7.97 (8H, ArH).

Step E: (E)-1-[2-[4-(2-chloro-ethoxy)-phenyl]-1 -(4-methoxy-phenyl)-propenyl]-6-methoxynaphthalene

1-lodo-6-methoxynaphthalene (3.38 g, 11.9 mmol) dissolved in THF (50 ml) was treated with n-butyllithium (7.8 ml, 12.5 mmol) at −78° C. A solution of the compound from step D (3.8 g, 11.9 mmol) in THF (40 ml) was added and the resulting mixture stirred at −78° C. for 2 h followed by an additional 19 h at room temperature. The reaction mixture was quenched with saturated NH₄Cl (10 ml) and washed with brine (60 ml) and water (2×60 ml). The crude product was chromatographed on silica gel with hexane/ethyl acetate (10:1) to yield 1.28 g (23.4%) of (E)-1-[2-[4-(2-chloro-ethoxy)-phenyl]-1-(4-methoxy-phenyl)-propenyl]-6-methoxy-naphthalene as a white solid and 0.95 g (17.4%) of a mixture of (E/Z)-1-[2-[4-(2-chloro-ethoxy)-phenyl]-1-(4-methoxy-phenyl)-propenyl]-6-methoxy-naphthalene. ¹H-NMR (400 MHz, CDCl₃) δ_{H} (E)-isomer 1.88 (3H, s, CH₃), 3.71 (3H, s, OCH₃), 3.85 (2H, t, ClCH₂), 3.97 (3H, s, OCH₃), 4.25 (2H, t, OCH₂), 6.57-8.01 (14H, ArH). (Z)-isomer 2.35 (3H, s, CH₃), 3.71(2H, t, ClCH₂), 3.80 (3H, s, OCH₃), 3.90 (3H, s, OCH₃), 4.07 (2H, t, OCH₂), 6.52 -7.91 (14H, ArH).

Step F: (E)-5-[2-[4-(2-chloroethoxy)phenyl]-1-(4-hydroxyphenyl)-propenyl]-naphthalen-2-ol

The E/Z mixture from Step E (0.59 g, 1.3 mmol) was dissolved in CH₂Cl₂ (50 ml) under Ar and cooled to −78° C. A solution of BBr₃ in CH₂Cl₂ (1.0 M, 17.5 ml, 17.5 mmol) was slowly added under Ar at −78° C. The reaction mixture was stirred at room temperature for 15 h and treated with water (5 ml). The aqueous layer was extracted with CH₂Cl₂ (2×30 ml), washed with 20% NaHCO₃ (30 ml) and water (30 ml). The crude product was chromatographed on silica gel with 3% MeOH/CH₂Cl₂ to give (E)-5-[2-[4-(2-chloroethoxy)phenyl]-1 -(4-hydroxyphenyl)-propenyl]-naphthalen-2-ol (0.07 g, 12%) and a mixture of (E/Z)-5-[2-[4-(2-chloroethoxy)phenyl]-1-(4-hydroxyphenyl)-propenyl]-naphthalen-2-ol (0.27 g, 48%). ¹H-NMR (400 MHz, CDCl₃) δ_{H} (E)-isomer 1.88 (3H, s, CH₃), 3.85 (2H, t, ClCH₂), 4.25 (2H, t, OCH₂), 6.49-8.00 (14H, ArH). (Z)-isomer 2.34 (3H, s, CH₃), 3.72 (2H, t, ClCH₂), 4.06 (2H, t, OCH₂), 6.52-7.90 (14H, ArH).

Step G: (E)-5-[1 -(4-hydroxyphenyl)-2-[4-(2-pyrrolidin-1-yl-ethoxy)-phenyl]-propenyl]-naphthalen-2-ol

To (E)-5-[2-[4-(2-chloroethoxy)phenyl]-1 -(4-hydroxyphenyl)-propenyl]-naphthalen-2-ol (54 mg, 0.125 mmol) (from Step F), dissolved in ethanol (2 ml), was added pyrrolidine (0.5 ml). The mixture was sealed and heated at 105° C. for 15 h while stirring. The solvents were removed and the residue chromatographed on silica gel with 7% MeOH/CH₂Cl₂ to give 40 mg (68%) of the title compound. ¹H-NMR (400 MHz, DMSO-d6) δ_{H} 1.76 (3H, s, CH₃), 1.86 (4H, m, NCH₂CH₂), 2.78 (4H, m, NCH₂CH₂), 3.02 (2H, m, NCH₂CH₂O ), 4.03 (2H, t, OCH₂), 6.42-7.87 (14H, ArH), 9.19 (1H, brs, OH), 9.75 (1H, brs, OH).

### EXAMPLE 4

### Alternative Method for the Preparation of (E/Z)-5-[1-(4-hydroxyphenyl)-2-[4-(2-pyrrolidin-1-yl-ethoxy)-phenyl]-propenyl]-naphthalen-2-ol

Step D-1: 1-(4-methoxyphenyl)-2-[4-(2-pyrrolidin-1-yl-ethoxy)-phenyl]-propan-1-one

A mixture of the compound from Step C (7.2 g, 28 mmol) and K₂CO₃ (9.5 g, 69 mmol) in DMF (50 ml) was heated to 100° C. under Ar. 1-(2-chloroethyl)pyrrolidine hydrochloride (5.7 g, 33 mmol) was added in portions over 10 min and the reaction mixture heated for an additional 1.5 h at 100° C. After cooling, the crude reaction mixture was filtered and the DMF removed from the filtrate by evaporation. The solid residue obtained from the filtration step was extracted twice with ethyl acetate (100 ml) and concentrated. The residue so obtained was added to the oil concentrate obtained from the filtrate. The combined crude product was washed with brine, the solvent evaporated and purified by chromatography on silica gel using 7% CH₃OH/CH₂Cl₂ to yield 7.4 g (75%) of the title compound. ¹H-NMR (400 MHz, DMSO-d6) δ_{H} 1.36 (3H, d, CH₃), 1.67 (4H, m NCH₂CH₂), 2.51 (4H, m NCH₂CH₂), 2.76 (2H, t, NCH₂CH₂O), 3.81 (3H, s, OCH₃), 4.00 (2H, t, NCH₂CH₂O), 4.85 (1H, q, CH), 6.85-7.97 (8H, ArH).

Step E-1: (E/Z)-1-(2-[4-[2-(6-methoxy-naphthalen-1-yl)-2-(4-methoxyphenyl)-1-methyl-vinyl]-phenoxy]-ethyl)-pyrrolidine.

1-lodo-6-methoxy-naphthalene (4.0 g, 14.2 mmol) dissolved in THF (20 ml), was treated with n-butyllithium (0.9 g, 14.1 mmol) at −78° C. A solution of the compound from step D-1 (5.0 g, 14.1 mmol) in THF (25 ml) was added and the resulting mixture stirred at −78° C. for 1.5 h followed by an additional 5 days at room temperature. The crude product, dissolved in ethanol (60 ml) was treated with 30% HCl (20 ml) and refluxed for 3 h to yield 2.76 g (40%) of the title compound as a mixture of E and Z isomers.

Step F-1: (E/Z)-5-[1-(4-hydroxyphenyl)-2-[4-(2-pyrrolidin-1-yl-ethoxy)-phenyl]-propenyl]-naphthalen-2-ol

To a solution of (E/Z)-1-(2-[4-[2-(6-methoxy-naphthalen-1-yl)-2-(4-methoxy-phenyl)-1-methyl-vinyl]-phenoxy]-ethyl)-pyrrolidine (2.5 g, 5.0 mmol), from Step E-1, dissolved in CH₂Cl₂ (200 ml) and cooled to −78° C. under Ar, was slowly added at −78° C. under Ar, an in CH₂Cl₂ diluted solution of BBr₃ (8 ml, 79.8 mmol). The reaction mixture was stirred overnight at room temperature and then treated with water (250 ml). The crude product was chromatographed on silica gel using 7% CH₃OH/CH₂Cl₂ to yield 2.0 g (72%) of the title compound as a mixture of E/Z isomers.

### EXAMPLE 5

### Effectiveness of (E)-5-[1-(4-hydroxyphenyl)-2-[4-(2-pyrrolidin-1-yl-ethoxy)-phenyl]-propenyl]-naphthalen-2-ol Against MCF-7 Cancer Cell Proliferation

Those skilled in the art will appreciate that several acceptable estrogen receptor-binding assays are known and available for initial screening of the compounds of the present invention. The initial screen chosen for detecting anti-estrogenic/estrogenic activity was a human cell line assay, namely the MCF-7 cell proliferation assay. MCF-7 cells are estrogen-receptor positive (ER+) cancer cells that respond to estradiol stimulation. Anti-estrogenic activity is measured in terms of a test article's ability to inhibit estradiol stimulated proliferation, implying an antagonistic action on the estrogen receptor and estrogenic activity can be inferred from increased proliferation. The following testing procedure was used.

MCF-7 cells were maintained in a RPMI medium, free of phenol red, and supplemented with 5% charcoal-stripped foetal calf serum, hydrocortisone, bovine insulin, penicillin and streptomycin until they reached 70% confluence. The cells were kept in a 5% CO₂ atmosphere and, prior to treatment, were washed twice with Ca⁺⁺/Mg⁺⁺ free Hanks balanced salt solution and harvested with 1 mM EDTA prepared in Ca⁺⁺/Mg⁺⁺ free Hanks balanced salt solution. After the washes the cells were re-suspended in medium. Cells were seeded in 96-well plates and incubated for 16 hours in 5% charcoal-stripped calf serum phenol red-free medium. Cells were then treated continuously with estradiol, a test article, or a combination of both using various serum concentrations. Cell survival was evaluated after 3-6 days, by replacing the culture media with 150 μl of fresh medium containing 10 mM 4-(2-hydroxyethyl)-1-piperazineethamsulfonic acid buffer (pH 7.4) followed by the addition of 50 μl of 2.5 mg/ml of 3-(4,5-dimethylthiazo-2-yl)-2,5-diphenyltetrazolium bromide (MTT). After 4 hours of incubation at 37° C. the medium and MTT were removed and 200 μl of dimethylsulfoxide (DMSO) was added to dissolve the precipitate of reduced MTT, followed by the addition of 25 μl of glycine buffer (0.1M glycine plus 0.1M NaCl, pH 10.5). Plates were shaken for 15 minutes and the absorbance was determined at 570 nm with a microplate reader (BIORAD, model 450). The data is expressed as percent (%) cell growth in comparison with untreated cells.

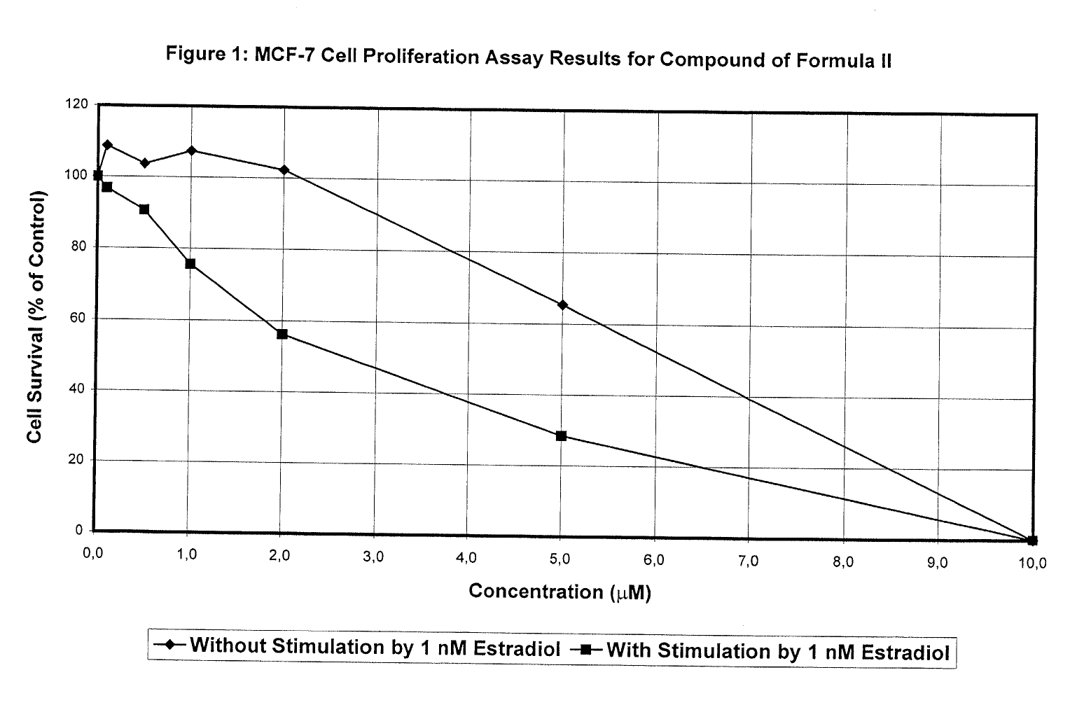

FIG. 1 shows the dose-response curves of estradiol stimulated versus unstimulated MCF-7 cells in the presence of the compound depicted by Formula II. The shift in the response curve is an indication that the test compound is antagonizing the effect of estradiol on these cells. This is a positive indication that the compounds of the present invention are of potential in the treatment of a wide variety of disease states involving the estrogen receptor.

### EXAMPLE 6

### Effectiveness of (E)-5-[1-(4-hydroxyphenyl)-2-[4-(2-pyrrolidin-1-yl-ethoxy)-phenyl]-propenyl]-naphthalen-2-ol Against Endothelial Cells and Epithelial Cancer Cell Lines

Those skilled in the art will appreciate that several acceptable cell proliferation assays are known and available for demonstrating the activity of the compounds of the present invention. The proliferation of endothelial cells is an important step in the process of blood vessel formation. Therefore, cells derived from the endothelium are useful in the study of angiogenesis and in vitro model systems utilizing endothelial cells have the additional advantage of simplicity. Two model endothelial cell lines are the Human Umbilical Vein Endothelial Cells (HUVEC) and the Bovine Brain-derived Capillary Endothelial Cells (BBCE). Both have been used extensively to study the biology of endothelial cells. The following testing procedures were used.

Bovine Brain-derived Capillary Endothelial Cells (BBCE)

BBCE are maintained in a regular medium containing DMEM plus 10% new-born calf serum and 2.5 mg/ml bFGF is added every second day. Sub-confluent cells are collected, diluted at 5,000 cells per ml, and seeded in 1 ml aliquots per well into 12-well cluster dishes. Cells are treated with the drug candidates or the vehicle every second day. 2-Methoxyestradiol is used as a positive control. Cells are washed and counted using a Coulter particle counter after six days. The results are expressed as IC₅₀ values, that is the concentration of the respective test compound resulting in half the number of cells that are obtained with untreated cells (controls).

Cancer Cells (MCF-7; MDA-MB-435; HCT-116; B16) & Human Umbilical

Vein Endothelial Cells (HUVEC) HUVEC are maintained in M199 medium supplemented with 90 mg/ml heparin, 2mM L-glutamate, 10% foetal bovine serum (FBS), 90 mg/ml heparin sulphate, 20 mg/ml endothelial cell growth supplement, 100 mg/ml penicillin and 100 mg/ml streptomycin. The MCF-7, MDA-MB-435, HCT-116 and B16 cells are cultured in RPMI, D-MEM, McCoy's 5R medium and RPMI medium supplemented with 10% glutamine, 1% non-essential amino acids (10 mM) and 1% sodium pyruvate (100 mM) respectively. All culture mediums are supplemented with 10% FBS. All cells are maintained in an atmosphere of 5% CO₂. Exponentially growing cells are seeded in 96-well plates and incubated for 16 hours. The cells are then treated continuously with the test articles. Cell survival is evaluated 96 hours later, by replacing the culture media with 150 μl of fresh medium containing of 10 mM 4-(2-hydroxyethyl)-1-piperazineethanesulfonic acid buffer, (pH 7.4). Next, 50 μl of 2.5 mg/ml of 3-(4,5-dimethylthiazo-2-yl)-2,5-diphenyltetrazolium bromide (MTT) in phosphate buffer solution (PBS), pH 7.4, is added. After 3-4 hours of incubation at 37° C., the medium and MTT are removed and 200 μl of dimethylsulfoxide (DMSO) is added to dissolve the precipitate of reduced MTT, followed by the addition of 25 μl glycine buffer (0.1M glycine plus 0.1M NaCl, pH 10.5). The absorbance is determined at 570 nm with a microplate reader (BIORAD).

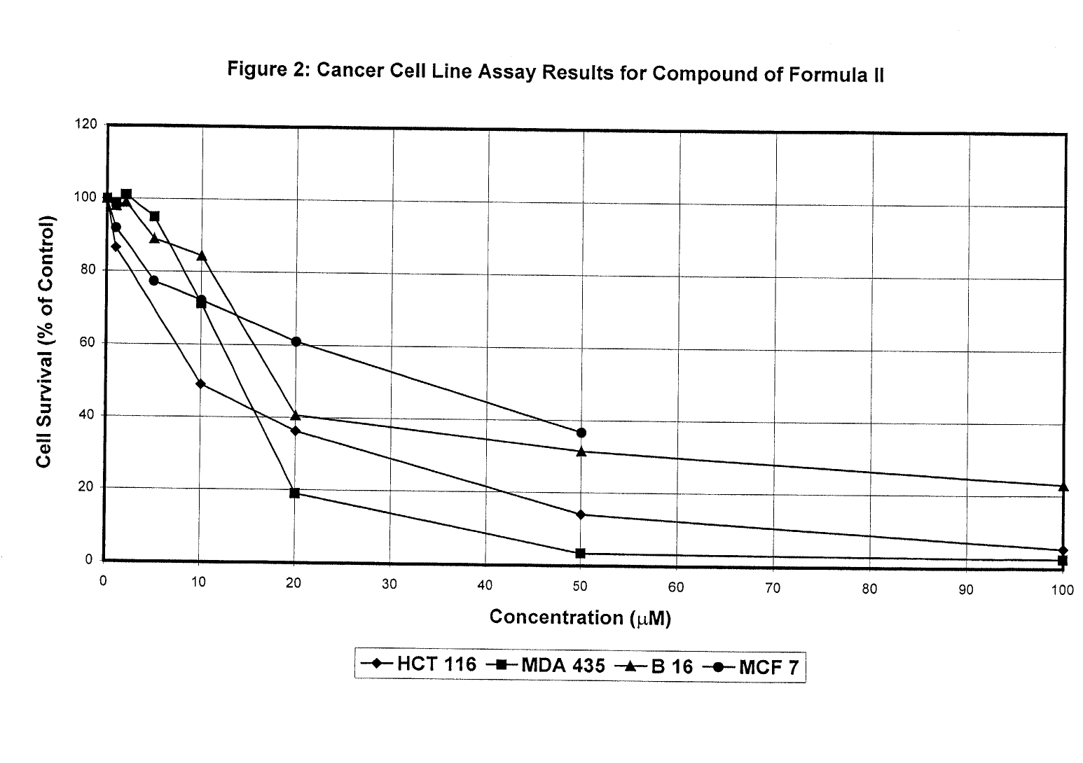

FIG. 2 shows the effects of the compound depicted by Formula II on the proliferation of four cancer cell lines. This is a positive indication that the compounds of the present invention are of potential in the treatment of a wide variety of cancers.

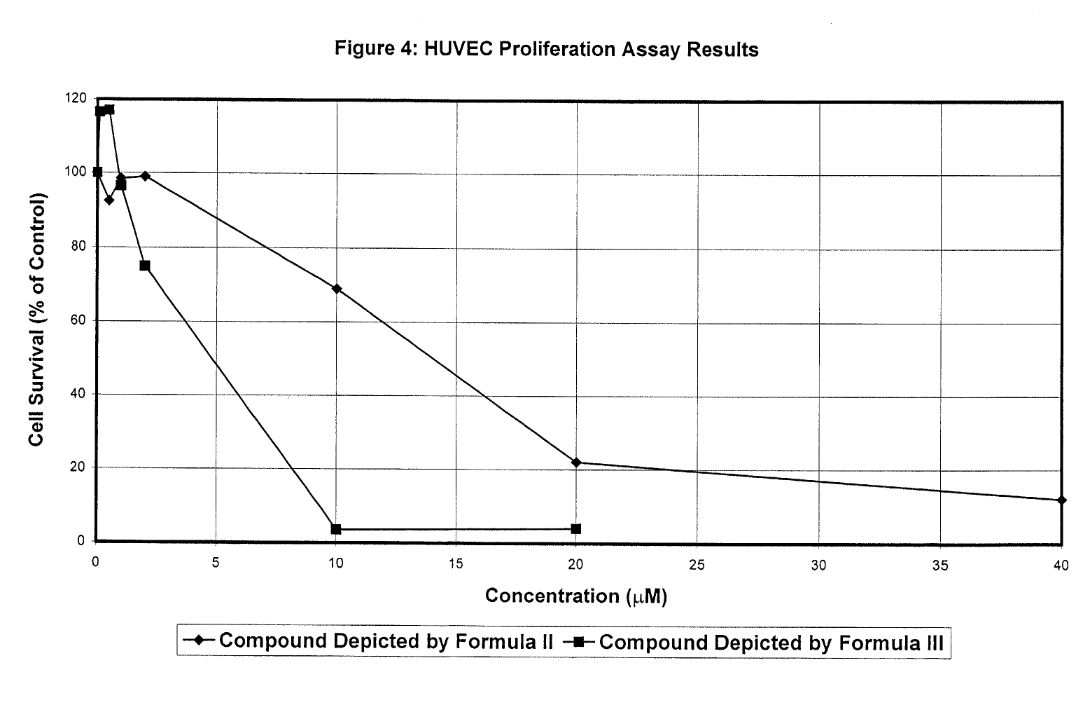

FIG. 4 shows the effects of the compound depicted by Formula II on the proliferation of HUVEC cells. This is an indication that the test compounds of the present invention are of potential in the treatment of diseases characterized by the undesired proliferation of endothelial cells.

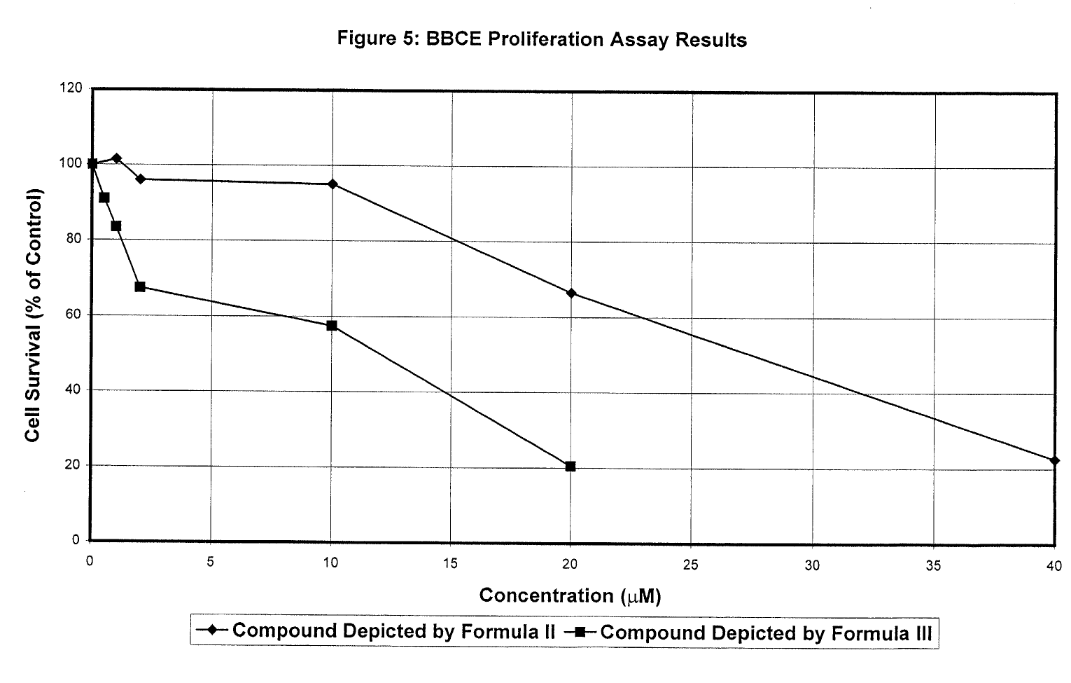

FIG. 5 shows the effects of the compound depicted by Formula II on the proliferation of BBCE cells. This is also an indication that the test compounds of the present invention are of potential use for the treatment of diseases characterized by the undesired proliferation of endothelial cells.

### EXAMPLE 7

### Effectiveness of (E/Z)-1-(2-[4-[2-(6-methoxy-naphthalene-1-yl)-2-(4-methoxy-phenyl)-1-methyl-vinyl]-phenoxy]-ethyl-pyrrolidine Against Endothelial Cells and Epithelial Cancer Cell Lines

The compound depicted by Formula III was tested using the same procedures as disclosed in Example 4 above.

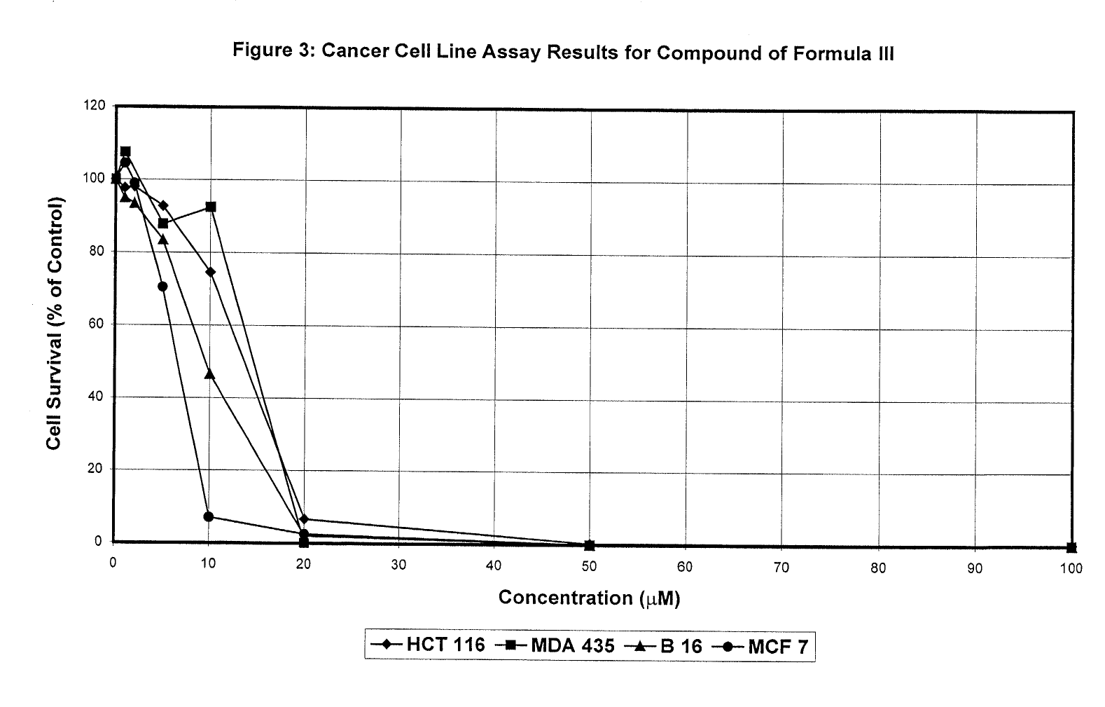

FIG. 3 shows the effects of the compound depicted by Formula III on the proliferation of four cancer cell lines. This is a positive indication that the compounds of the present invention are of potential in the treatment of a wide variety of cancers.

FIG. 4 shows the effects of the compound depicted by Formula III on the proliferation of HUVEC cells. This is an indication that the test compounds of the present invention are of potential in the treatment of diseases characterized by the undesired proliferation of endothelial cells.

FIG. 5 shows the effects of the compound depicted by Formula III on the proliferation of BBCE cells. This is also an indication that the test compounds of the present invention are of potential in the treatment of diseases characterized by the undesired proliferation of endothelial cells.

### EXAMPLE 8

### Effectiveness of (E)-5-[1-(4-hydroxyphenyl)-2-[4-(2-pyrrolidin-1-yl-ethoxy)-phenyl]-propenyl]-naphthalen-2-ol at Displacing Estrogen ER-Alpha and ER-Beta in Human Recombinant Estrogen Receptors

The human estrogen receptor occurs in two subtypes, alpha and beta. The stable expression of these individual receptor subtypes in cells provides a rapid and accurate means of quantifying the direct interaction of a test article with the estrogen binding sites. Briefly, this assay is conducted in 96 well plates where a series of concentrations of the test article are used to displace tritiated estradiol from either estrogen receptor alpha or estrogen receptor beta bearing cell membranes, under equilibrium conditions. The measurement of the displaced tritiated estradiol allows the determination of an IC₅₀ value (concentration of test article that inhibits 50% of the estradiol binding). This measurement is the primary test for mediation of the estrogen receptor and can also be used to measure the relative selectivity of the test article for either the alpha or beta subtype.

The alpha subtype assay measures the binding of [³H] Estradiol to the human recombinant estrogen receptor. The receptor preparation was obtained from PanVera Corporation and was used in an assay that followed the method taught by Obourn et. al. (Biochemistry, 1993; 6229-6236) with some minor variations. Briefly, after proper dilution, a 4.5 ng aliquot of receptor protein in modified Tris-HCL pH 7.5 buffer is incubated with 0.5 nM [³H] Estradiol for 2 hours at 25° C. Non-specific binding is estimated in the presence of 1.0 μM diethylstilbestrol. Membranes are filtered and washed 3 times, and the filters are counted to determine [³H] Estradiol specifically bound. Under the same conditions the receptor protein is incubated with varied concentrations of test article, ranging from 1 nM to 1 μM, and the displacement of [³H] Estradiol is measured in duplicate. The measurement of the displaced tritiated estradiol allows the determination of an IC₅₀ value, a direct measure of the test articles' interaction with the estrogen receptor alpha.

The beta subtype assay also allowed the determination of an IC₅₀ value for the test article under the same conditions as for the alpha subtype assay, with the exception that a 7.5 ng aliquot of receptor protein preparation was used.

Table 1 shows the IC₅₀ results of the compound depicted by Formula II. The IC₅₀ values are both below 100 nM. This is an indication of a very high binding affinity for the estrogen receptor and of the potential of the class of compounds represented by Formula I in the treatment of disease states involving the estrogen receptor.

TABLE 1
Estrogen Receptor Binding Assay Results

| | Estrogen Receptor | |
|---|---|---|
| Compound | Subtype | IC₅₀ Value |
| Formula II | Alpha | 5.54 nM |
| DES | Alpha | 0.923 nM |
| Formula II | Beta | 75.0 nM |
| DES | Beta | 2.44 nM |

Although the present invention has been described hereinabove by way of preferred embodiments thereof, it can be modified, without departing from the spirit and nature of the subject invention as defined in the appended claims.

1. A compound of Formula I comprising an ethene backbone, and A, B, C and D rings, or a pharmaceutically acceptable salt or ester thereof, 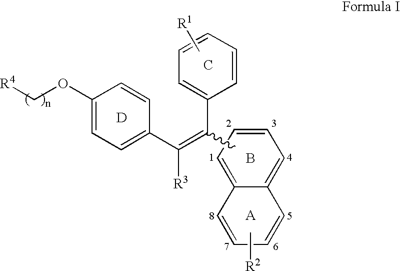
wherein:
i) R¹ represents a substituent selected from the group consisting of: hydroxyl, methoxy, ethoxy and esters;
ii) R represents a substituent selected from the group consisting of: hydroxyl, methoxy, ethoxy and esters;
iii) R₃ represents a substituent selected from the group consisting of: hydrogen, methyl, ethyl and cyano;
iv) R⁴ represents a substituent selected from the group consisting of: 1-pyrrolidinyl, 1-piperidinyl, methyl-1-pyrrolidinyl, dimethyl-1-pyrrolidinyl, 4-morpholino, dimethylamino, diethylamino, diisopropylamino and 1-hexamethyleneimino;
v) “n” is an integer from 1 to 4;
vi) said B-ring is connected at the 1-position or the 2-position to said ethene backbone;
vii) said R¹ substituent is located at the ortho, meta or para-position on said C-ring;
viii) said R² substituent is located at either the 5, 6, 7 or 8-position on said A-ring, and
ix) said ethene backbone has the E-configuration or the Z-configuration with respect to said B-ring and said D-ring.

2. The compound of claim 1 or a pharmaceutically acceptable salt or ester thereof
wherein:
i) R¹ is a hydroxyl group;
ii) R² is a hydroxyl group;
iii) R³ is a methyl group;
iv) R⁴ is a pyrrolidine group;
v) “n” is 2;
vi) R¹ is located at the para-position on said C-ring;
vii) R² is located at the 6-position on said A-ring, and
viii) said B-ring is linked to said ethene backbone at the 1-position;
ix) said ethene backbone has the E-configuration or the Z-configuration with respect to said B-ring and said D-ring.

3. The compound of claim 1 or a pharmaceutically acceptable salt or ester thereof
wherein:
i) R¹ is a methoxy group;
ii) R² is a methoxy group;
iii) R³ is a methyl group;
iv) R⁴ is a pyrrolidine group;
v) “n” is 2;
vi) R¹ is located at the para-position on said C-ring;
vii) R² is located at the 6-position on said A-ring;
viii) said B-ring is linked to said ethene backbone at the 1-position; and
ix) said ethene backbone has the E-configuration or the Z-configuration with respect to said B-ring and said D-ring.

4. The compound of claim 2, having the following formula:

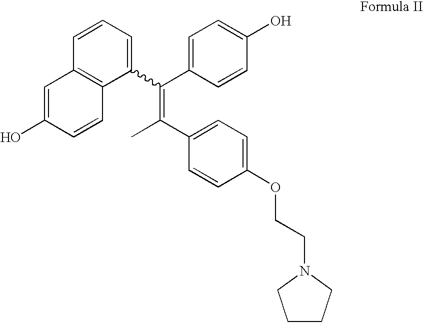

5. The compound of claim 3, having the following formula:

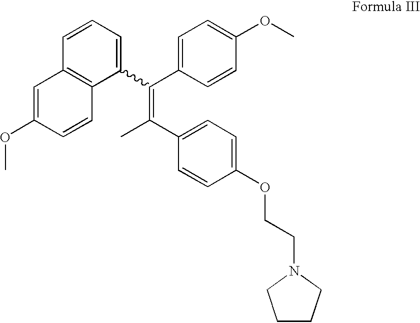

6. A process for the preparation of a compound of Formula I, said process comprising:
(a) reacting a molecule of Formula 1.12 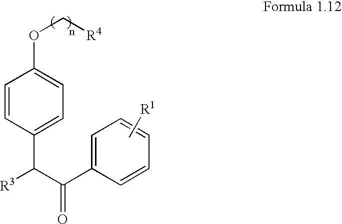
wherein:
i) R¹ represents a substituent selected from the group consisting of: hydroxyl, methoxy, ethoxy and esters;
ii) R³ represents a substituent selected from the group consisting of: hydrogen, methyl, ethyl and cyano;
iii) R⁴ represents a substituent selected from the group consisting of: 1-pyrrolidinyl, 1-piperidinyl, methyl-1-pyrrolidinyl, dimethyl-1-pyrrolidinyl, 4-morpholino, dimethylamino, diethylamino, disopropylamino and 1-hexamethyleneimino;
iv) “n” is an integer from 1 to 4;
v) said R¹ substituent is located at the ortho, meta or para-position;
with a reagent mixture composed of 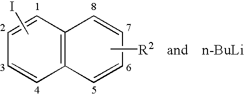
wherein:
i) R² represents a substituent selected from the group consisting of: hydroxyl, methoxy, ethoxy and esters;
ii) I is located at the 1- or 2-position, and
iii) R² is located at the 5, 6, 7 or 8-position;
thereby generating a reaction mixture;
(b) treating said reaction mixture with hydrochloric acid hence generating an acidified reaction mixture; and
(c) recovering said compound of Formula I from said acidified reaction mixture.

7. A process for the preparation of a compound of Formula II, said process comprising:
(a) reacting molecule of Formula 1.5 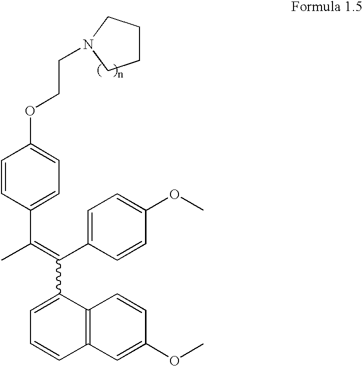
wherein n=1
with a reagent mixture composed of concentrated HBr in acetic acid or BBr₃
thereby generating a reaction mixture;
(b) recovering said compound of Formula II from said reaction mixture.

8. A process for the preparation of a compound of Formula II, said process comprising:
(a) reacting a molecule of Formula 1.8 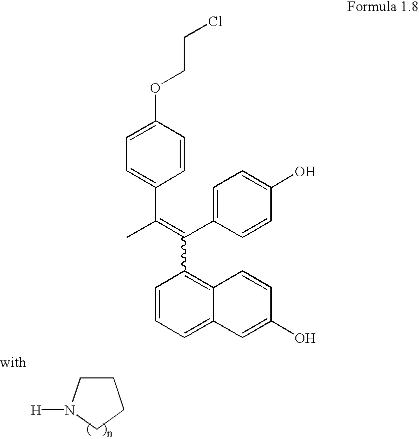
wherein n=1
thereby generating a reaction mixture
(b) recovering said compound of Formula 2 from said reaction mixture.

9. A process for the preparation of a compound of Formula III, said process comprising: (a) reacting a molecule of Formula 1.4 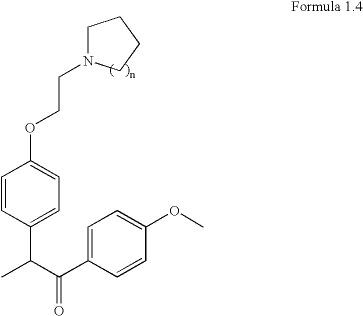
wherein n=1
with a reagent mixture composed of 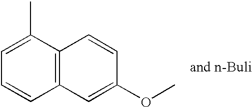
thereby generating a reaction mixture;
(b) treating said reaction mixture with hydrochloric acid hence generating an acidified reaction mixture; and
(c) recovering said compound of Formula III from said acidified reaction mixture.

10. A pharmaceutical composition comprising the compound of Formula I, II, III or a pharmaceutically acceptable salt or ester thereof, and at least one pharmaceutically acceptable carrier.

11. A process for the preparation of an anti-cancer agent of Formula I, said process comprising:
(a) reacting a molecule of Formula 1.12 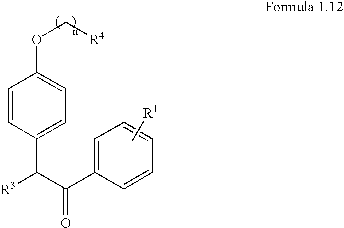
wherein
i) R¹ represents a substituent selected from the group consisting of: hydroxyl, methoxy, ethoxy and esters;
ii) R³ represents a substituent selected from the group consisting of: hydrogen, methyl, ethyl and cyano;
iii) R⁴ represents a substituent selected from the group consisting of: 1-pyrrolidinyl, 1-piperidinyl, methyl-1-pyrrolidinyl, dimethyl-1-pyrrolidinyl, 4-morpholino, dimethylamino, diethylamino, diisopropylamino and 1-hexamethyleneimino;
iv) “n” is an integer from 1 to 4, and
v) said R¹ substituent is located at either the ortho, meta or para-position;
with a reagent mixture composed of 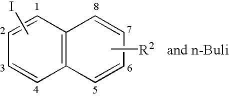
wherein:
i) R² represents a substituent selected from the group consisting of: hydroxyl, methoxy, ethoxy and esters;
ii) I is located at the 1- or 2-position,
iii) R² is located at the 5, 6, 7 or 8-position;
thereby generating a reaction mixture,
(b) treating said reaction mixture with hydrochloric acid, hence generating an acidified reaction mixture; and
(c) recovering said anti-cancer agent of Formula I from said acidified reaction mixture.

12. A process for the preparation of an anti-cancer agent of Formula II, said process comprising:
(a) reacting a molecule of Formula 1.5 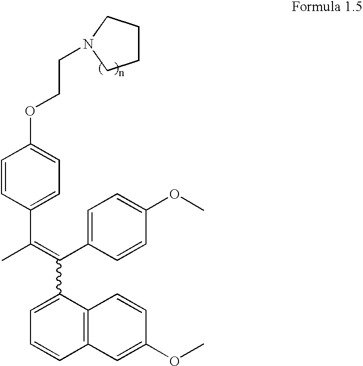
wherein n=1
with a reagent mixture composed of HBr in acetic acid or BBr₃, thereby generating a reaction mixture;
(b) recovering said anti-cancer agent of Formula II from said reaction mixture.

13. A process for the preparation of an anti-cancer agent of Formula II, said process comprising:
(a) reacting a molecule of Formula 1.8 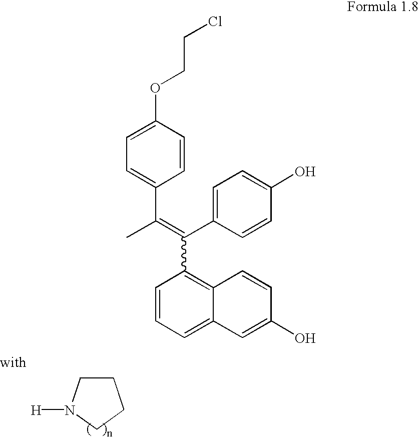
wherein n=1
thereby generating a reaction mixture, and
(b) recovering said anti-cancer agent of Formula II from said reaction mixture.

14. A process for the preparation of an anti-cancer agent of Formula III, said process comprising:
(a) reacting a molecule of Formula 1.4 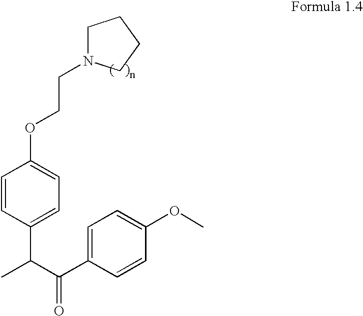
wherein n=1
with a reagent mixture composed of 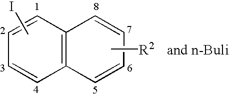
thereby generating a reaction mixture;
(b) treating said reaction mixture with hydrochloric acid hence generating and acidified reaction mixture; and
(c) recovering said anti-cancer agent of Formula III from said acidified reaction mixture.

15. A method of treating cancer comprising administering a therapeutically effective amount of a compound of Formula I to a patient in need thereof.
